(19) United States
(12) Patent Application Publication
(10) Pub. No.: US 2023/0363478 A1
(43) Pub. Date: Nov. 16, 2023

(54) Patterned Engagement Structure for Compact Adhesive Face Masks

(71) Applicant: Sepala LLC, Seattle, WA (US)

(72) Inventor: Benjamin Spencer, Oak Harbor, WA (US)

(73) Assignee: Sepala LLC, Seattle, WA (US)

(21) Appl. No.: 18/030,767

(22) PCT Filed: Oct. 6, 2021
(86) PCT No.: PCT/US2021/053854

§ 371 Date: Apr. 6, 2023

Related U.S. Application Data
(60) Provisional application No. 63/088,354, filed on Oct. 6, 2020.

(51) Int. Cl. A41D 13/11

(52) U.S. Cl. CPC A41D 13/1176; A41D 2300/328

(57) ABSTRACT

A face mask, comprising: a left half and a right half; one or more adhesive portions; and one or more release-coating portions; wherein the left half and right half are divided by a center axis; wherein the adhesive portions are strips located along an outer portion of the left half and the right half; wherein the face mask comprises a symmetrical folded configuration and an unfolded configuration; wherein the left half and the right half are configured to releasably engage one another in the symmetrical folded configuration; wherein the left half and the right half are configured to releasably engage a wearer's face in the unfolded configuration.

### CROSS-REFERENCE TO RELATED APPLICATIONS

This application claims priority and benefit to U.S. Provisional Application No. 63/088,354, filed on Oct. 6, 2020, titled Compact Adhesive Face Mask With And Without Release Liner; the contents of which are incorporated herein by this reference as though set forth in their entirety.

### FIELD OF USE

The invention is an innovation in the field of engagement structures for protective face masks.

### BACKGROUND

Face masks help prevent the spread of disease, provide protection from ambient air pollution, and prevent the inhalation of airborne toxins. The majority of protective face masks are attached via straps, fit relatively loosely, and generally do not create a seal tightly against a user's face. As a result, pollutants and pathogens can enter or exit from a mask's perimeter, compromising the mask's efficacy as a protective barrier. A select number of face masks have attempted to address this challenge through the use of adhesives for facial attachment, which attempt to create a seal between the mask and a person's face.

Despite their ability to provide a seal, existing adhesive face masks suffer from several shortcomings. First, they are often relatively bulky, difficult to store and carry (e.g., in wallets, pockets, or purses), and difficult to package in thin/compact containers. Second, their interior surface(s)—on the inside of the mask, worn close to a person's face—are not completely encapsulated and sealed off from external elements before use. This makes them potentially susceptible to contamination. Third, their application and removal often involve multiple tedious steps. This makes them difficult to apply and wear. Fourth, once removed, the release liners typically used to protect masks' adhesive are difficult to store and to subsequently re-attach to adhesive masks—this hinders the repeated use of adhesive masks. Fifth, it is often difficult to reseal adhesive masks and encapsulate contaminated interior surfaces following use/prior to disposal. Sixth, adhesive masks often require specialized manufacturing processes that complicate their production and/or increase the costs of production equipment. Finally, adhesive masks are often composed of materials that are derived from non-renewable and/or non-recycled sources that do not biodegrade easily and/or are difficult to recycle upon disposal. As such, they have negative environmental impacts.

Accordingly, what is needed is an engagement structure for adhesive masks that allows for easier storage; enhanced sealing from contaminants; application and removal that is more efficient, user-friendly and intuitive; repeated usage; resealing and encapsulation of contaminated interior surfaces; and improved manufacturing—all while being readily reusable.

### SUMMARY

The following presents a simplified overview of the example embodiments in order to provide a basic understanding of some embodiments of the present disclosure. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

Various embodiments of the present disclosure may be directed to an engagement structure for an adhesive face mask. The adhesive face mask may be self-contained and require no additional packaging, or tethering mechanisms for storage and repeated use.

In one embodiment, the novel compact adhesive face mask may comprise two single or multi-ply layers of air filtering material. The mask may have a shape roughly similar to a lens, oval, diamond, rhombus, parallelogram, triangle, or other shape with two primary edges consisting of curved or straight line(s). The air-filtering layers may be part of the same piece of continuous material or may be separate pieces that are connected to one another by adhesive or other means. A left edge of the mask may comprise both a release coating and a pressure sensitive adhesive (PSA) on the interior of the mask and a right edge may also comprise both a release coating and a PSA on the interior of the mask and opposite the left edge.

In a preferred embodiment, the PSA may be applied in band(s), shape(s), or pattern(s) (henceforth referred to as portions) that dovetail or otherwise correspond with portions of release coating. Thus, the corresponding portions of PSA and release coating on the interior face of one side of the mask may be mirrored and inverted on the opposing interior face of the mask's other side, such that when the interior faces of the mask are aligned and pressed together along the two opposite edges, the PSA portion on one interior face of the mask may coincide with and form a temporary bond with the release-coating portion on the other interior face of the mask. This creates a patterned engagement structure and an enclosed pocket inside the mask without the need for a release liner as an intermediate coupling material.

In some embodiments, the portions of the PSA and release coating may be similar, but not identical, sizes to create different effects. For example, in one embodiment, the PSA may form a portion that is slightly narrower or smaller than the corresponding release-coating portion, such that a weaker overall bond is created. In another embodiment, the PSA may form a portion that is slightly wider or bigger than the corresponding release-coating portion, such that a stronger overall bond is created.

In a separate embodiment, the mask may include one layer of a removable double-sided release liner or a double layer of single-sided release liner, sandwiched between two sides of the mask. The release liner may serve as an intermediate coupling material that temporarily bonds the PSA portions to one another along the mask's second edge. Users may pull apart the two sides of the mask to open it, remove the inner release liner if present, and adhere the mask to their face for protection from airborne contaminants and/or to prevent the spread of pathogens.

Novel and improvements of the mask over the prior art include but are not limited to: 1) an ultra-thin profile, making it easy to carry in wallets, pockets, pursues etc., to package in thin/compact containers and to dispense in large quantities; 2) an interior surface that is fully enclosed/encapsulated prior to use; 3) easier to apply and remove from a user's face; 4) may be re-enclosed following use/prior to reuse; 5) may be resealed upon disposal so as to encapsulate interior contaminates; and 6) simplified processes involved in adhesive face-mask manufacture. The mask may be constructed from a wide variety of air-filtering, release and adhesive materials, and adhesive materials so as to be optimized for single use or repeated use. The mask may be constructed from materials, such as paper or fabric, that are renewable, biodegradable, compostable, recycled, and/or recyclable.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The accompanying drawings, which are incorporated in and constitute a part of this specification, illustrate embodiments of the disclosure and together with the general description of the disclosure given above and the detailed description of the drawings given below, serve to explain the principles of the disclosure. In certain instances, details that are not necessary for an understanding of the disclosure or that render other details difficult to perceive may have been omitted.

FIG. 1 is an illustration of one embodiment of an adhesive face mask with a patterned engagement structure as disclosed herein.

FIG. 2 is an illustration of a stack of face masks in a folded configuration with a patterned engagement structure as disclosed herein.

FIG. 3 is an illustration of one embodiment of a face mask with a patterned engagement structure as disclosed herein.

FIG. 4 is an illustration of one embodiment of a face mask with a patterned engagement structure as disclosed herein.

FIG. 5 is an illustration of one embodiment of a face mask with a patterned engagement structure as disclosed herein.

FIG. 6 is an illustration of one embodiment of a transdermal patch with a patterned engagement structure as disclosed herein.

FIG. 7 is an illustration of one embodiment of a transdermal patch with a patterned engagement structure as disclosed herein.

FIG. 8 is an illustration of one embodiment of a transdermal patch with a patterned engagement structure as disclosed herein.

FIG. 9 is an illustration of one embodiment of an adhesive bandage with a patterned engagement structure as disclosed herein.

FIG. 10 is an illustration of one embodiment of a reusable tape with a patterned engagement structure as disclosed herein.

FIGS. 11A-11C are illustrations of embodiments of adhesive shoes with a patterned engagement structure as disclosed herein.

FIGS. 12A-12B are illustrations of a double-sided tape with a patterned engagement structure as disclosed herein

### DETAILED DESCRIPTION OF EMBODIMENTS

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As is used in the specification and the appended claims, the singular forms “a,” “an,” and “the” include plural referents unless the context clearly dictates otherwise. Ranges expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. In the following detailed description, numerous specific details are set forth in order to provide a thorough understanding of various aspects of one or more embodiments. However, these embodiments may be practiced without some or all of these specific details. In other instances, well-known methods, procedures, and/or components have not been described in detail so as not to unnecessarily obscure aspects of embodiments.

While multiple embodiments are disclosed, still other embodiments will become apparent to those skilled in the art from the following detailed description. As will be realized, these embodiments are capable of modifications in various obvious aspects, all without departing from the spirit and scope of protection. Accordingly, the screenshots, figures, and the detailed descriptions thereof, are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 15% of the indicated number or range of numbers. In one embodiment, the term “approximately” and “about”, refer to a deviance of between 0.0001-40% from the indicated number or range of numbers.

Embodiments of the present invention will now be described, by way of example, with reference to the accompanying drawings.

As used herein, PSA, or pressure sensitive adhesive, may be substituted for adhesive or substantially any other material configured to allow adhesion to another surface.

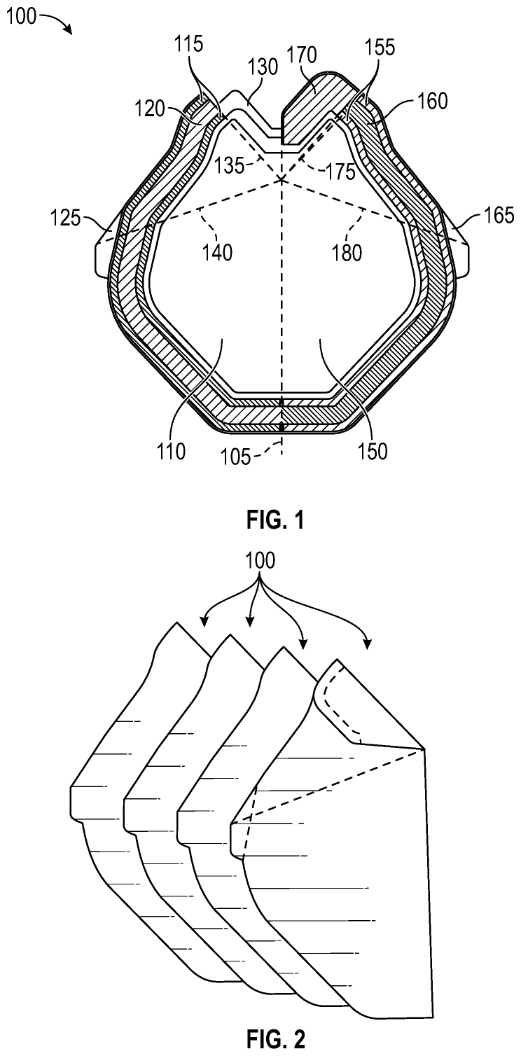

FIG. 1 is an illustration of one embodiment of an adhesive face mask with a patterned engagement structure as disclosed herein. As shown in FIG. 1, the adhesive face mask 100 may comprise an inner left half 110 and an inner right half 150. The left half and right half may be designated by a center axis 105. The left half 110 and the right half 150 may be substantially symmetrical in shape across the center axis 105.

The left half 110 may comprise left release-coating portions 115, a left PSA portion 120, and a left tab 125. The left PSA portion 120 may be placed between the left release-coating portions 115, wherein the combination of the left PSA portion 120 and the left release-coating portions 115 substantially follow the outside portion of the left half 110.

The right half 150 may comprise right PSA portions 155, a right release-coating portion 160, and a right tab 165. The right PSA portions 155 may be placed on both sides of the right release-coating portion 160, wherein the combination of the right PSA portions 155 and the right release-coating portion 160 substantially follow the outside portion, or an outer edge, of the right half 150.

In alternate embodiments, the PSA portions 120, 155 and release-coating portions 115, 160 may be substantially any shape, configuration, or coverage amount of the inside surface of the face mask.

When the face mask 100 is folded along the center axis 105, the left PSA portion 120 and right release-coating portion 160 may substantially overlap and/or engage one another, while the left release-coating portions 115 and the right PSA portions 155 may substantially overlap and/or engage one another. In a preferred embodiment, the release-coating portions 115, 160 may be wider than the PSA portions 120, 155 to which they engage when the face mask 100 is folded along the center axis 105, which may allow the PSA portions 120, 155 to more easily disengage and allow the face mask 100 to be unfolded. In other embodiments, the release-coating portions 115, 160 may be narrower than the PSA portions 120, 155 to which they engage when the face mask 100 is folded along the center axis 105.

When folded in this matter, the face mask 100 may form an enclosed pocket that may prevent contact of the face mask's 100 interior surfaces with potential contaminants before and after uses. This may allow for ease of safe storage for later use. In some embodiments, the face mask 100 may be folded in such a way that the various PSA portions 120, 155 engage one another rather than the corresponding release-coating portions 115, 160, such that a permanent or stronger engagement is made by the left half 110 and right half 150.

In one embodiment, the face mask 100 may comprise a single layer of filter material. In an alternate embodiment, the face mask 100 may comprise two (2) or more layers of material, with at least one layer comprising a filter material. In some embodiments the layers may be a filter material, or any other material known to be useful as components of face masks.

The left tab 125 and right tab 165 may be extensions of the material that makes up the face mask 100, and preferably contains no exposed adhesive, such that when the face mask 100 is folded, a user may easily grasp the tabs 125, 165 to unfold and open the face mask 100.

The face mask 100 may comprise an adhesive protrusion receiving portion 130 and an adhesive protrusion portion 170. The adhesive protrusion portion 170 and adhesive protrusion receiving portion 130 may be configured to engage one another and fold over and adhere to the outside surface of face mask 100 in order to provide the face mask 100 with a three-dimensional shape that is contoured to engage a user's face, specifically covering the user's mouth and nose.

The face mask may comprise several creases 135, 140, 175, 180 configured, in combination with a crease forming the center axis 105, to allow the face mask 100 to conform to a user's face in novel ways. The creases 135, 140, 175, 180 may be premade, or made by a user prior to wearing the face mask 100. In one embodiment, creases 135, 175 define the fold for enabling the nose—ridge configuration resulting from the adhesive protrusion receiving portion 130 and the adhesive protrusion portion 170, wherein the creases 135, 175 enable the adhesive protrusion receiving portion 130 and the adhesive protrusion portion 170 to fold over, adhere to the outside surface of the mask 100, and form the mask's nose—ridge configuration. The creases 140, 180 may function as control joints and enable the mask 100 to be more easily opened, facilitate creation of the mask's 3D-shape, and prevent convex “denting” in the mask when a user pulls the two sides apart during opening.

In some embodiments, the layers of the face mask 100 may be made of materials such as filter paper, film, or fabric. Such materials may comprise qualities such as filtration efficiency and breathability. The layers of the face mask 100 may also be made of materials such as low-porosity paper, film, or fabric. Such materials may serve as a substrate for release coatings and adhesives and/or to strengthen other layers of the face mask. In some embodiments, the face mask may comprise a barrier coat comprising a glue or glue with clay filler rather than, or in addition to, a low-porosity paper, film, or fabric. Such materials may also serve as a substrate for release coatings and adhesives and/or to strengthen layers of the face mask. For embodiments of a mask with two or more layers, the layers may be held together by glue or any methods known by one skilled in the art. In one embodiment, the release-coating portion may comprise material such as silicone, including thermal cure silicone, free-radical radiation cure silicone, or cationic radiation cure silicone. In one embodiment, the PSA may comprise characteristics such as biodegradable, skin-safe, synthetic rubber, and hot melt.

A preferred embodiment of the face mask 100 is a biodegradable or plastic-free and intended for public, or non-medical, use. In some embodiments, when worn properly, the face mask 100 may be intended to provide users with protection from air pollution, especially fine particulate matter (PM2.5). Some embodiments of the face mask 100 may be medical grade. In some embodiments, whether medical grade or non-medical grade, the face mask 100 may be made up of non-woven materials such as spunbond, meltblown, electrospun or similar non-woven fabrics. In some embodiments, these materials may be plastic-based and/or not biodegradable. Still, in other embodiments, whether medical grade or non-medical grade, the face mask 100 may be made from woven fabrics.

In a preferred embodiment, the face mask 100 may be soft enough to not make a sound while breathing, thick enough to hold a crease and fold, and strong enough to survive heavy exhalations such as coughing and sneezing or wetting in the rain.

Other materials considered for use with the face mask 100 include Twin Rivers Entex WS 36 CR BL (36 #/30005f), Little Rapids 7551-440-0 (44 #/30005f), Ahlstrom Munskjo 1278 hot oil filter paper and other filter papers, Glassine, Super Calendered Kraft, or other low porosity papers.

The release-coating portions may be made up other materials besides silicone, such as polysaccharide, starch, modified starch, modified starch ester, clay, or other biodegradable release agents. Specific possibilities for alternative, biodegradable, non-silicone release-coatings include Mayzo Escoat WPX and Ulterion 820 RC. The PSA may be made up of natural rubber-based (esp. deproteinized/hypoallergenic NR such as Yulex Pure, Vytex, Guayule-based NRs from American Biorubber or Bridgestone, or dandelion-based NRs from Kultevat), silicone-based, acrylate-based, synthetic rubber-based (other than GDI Bio BG H522), nano-PU-based, hydrocolloids, hydrogel, polylactic acid-based PSA, polyhydroxyalkanoate (PHA)-based PSA, polyhydroxybutyrate (PHB)-based PSAs, or poly(3-hydroxybutyrate-co-3-hydroxyhexanoate)(PHBH)-based PSA.

Still, other embodiments comprise coating and/or treatments of the mask that enhance the mask's performance, such as its filtration and biocidal capabilities. Such coating/treatments includes the addition of bioactive compounds (e.g., polyphenols, terpenoids, organic acids, polysaccharides); metal-containing compounds (e.g., silver); inorganic salts (e.g., NaCl, K₂SO₄, KCl); and composites of above (e.g., metal organic frameworks).

The face mask 100 may be manufactured multiple ways with different steps, different sequencing of steps, and different types of equipment. One approach to semi/automated manufacture involves discrete sheets of material that are processed using flatbed printing (screen printing, pad printing, inkjet printing) and die cutting technology. A second approach to semi/automated manufacture involves continuous/rolled webs of material that are processed using web converting machinery and rotary printing (flexographic printing, gravure printing, offset printing rotary screen printing, rotary hot melt screen printing) and rotary die scoring/creasing and cutting technology.

The steps involved in flatbed manufacturing processes and the web/rotary manufacturing processes are similar in many ways. However, each has its own advantages and disadvantages. Rotary processes are typically faster than flatbed processes. On the other hand, flatbed processes can open up possibilities that may be more difficult to achieve with rotary processes. For example, it is often easier to apply higher-viscosity release coatings using flatbed processes. Also, rotary equipment is expensive and is not readily available in many places. Flatbed equipment is more affordable and common worldwide.

Manufacturing the preferred embodiment of the mask involves two folds: a first fold along the mask's center line and a second subsequent fold along the mask's nose ridge. They can be manually folded or folded by automated folding processes such as knife folding and/or plow folding. As disclosed herein, the geometry of the preferred mask design includes straight parallel creases and edges to facilitate the control of mask orientation and travel during automated folding processes.

FIG. 2 is an illustration of a stack of face masks 100 in a folded configuration with a patterned engagement structure as disclosed herein. As shown in FIG. 2, the face masks 100 may be efficiently stacked and stored.

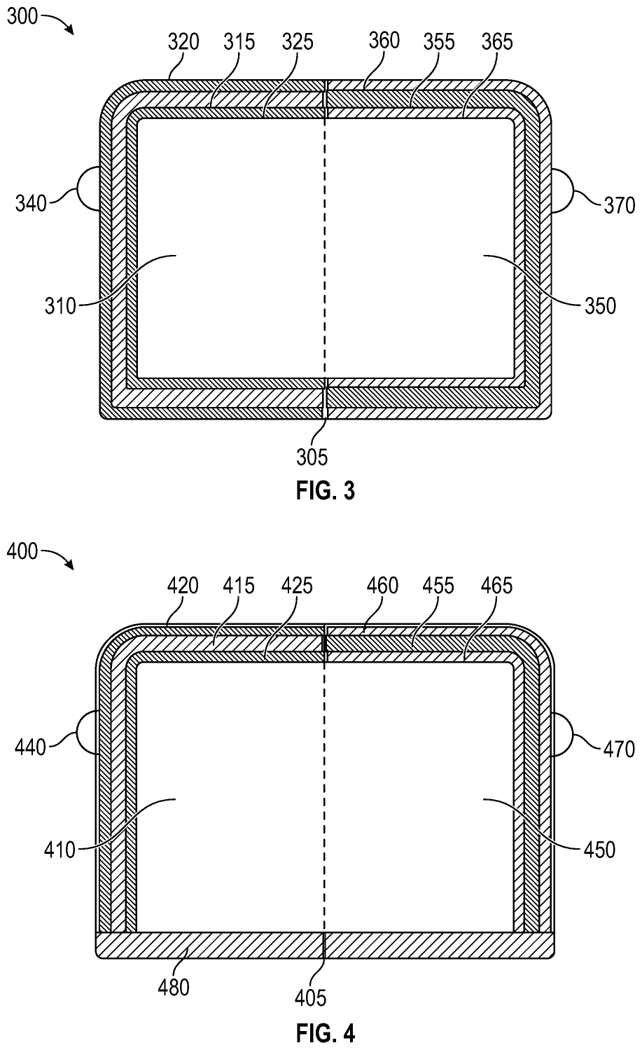

FIG. 3 is an illustration of one embodiment of a face mask with a patterned engagement structure as disclosed herein. As shown in FIG. 3, the face mask 300 may comprise a center axis 305, a left half 310, a right half 350, and tabs 340, 370. The PSA portions 315, 360, 365 and release-coating portions 320, 325, 355 of the face mask 300 may function substantially similarly to the PSA portions and release-coating portions of the face mask 100 shown in FIG. 1.

FIG. 4 is an illustration of one embodiment of a face mask with a patterned engagement structure as disclosed herein. As shown in FIG. 4, the face mask 400 may comprise a center axis 405, a left half 410, a right half 450, and tabs 440, 470. The PSA portions 415, 460, 465 and release-coating portions 420, 425, 455 of the face mask 400 may function substantially similarly to the PSA portions and release-coating portions of the face mask 300 shown in FIG. 3. Additionally, the face mask 400 may further comprise a lower PSA portion 480 along a lower strip of the face mask 400. The lower PSA portion 480 may provide a stronger bond between opposite halves of the mask along the mask's lower edge.

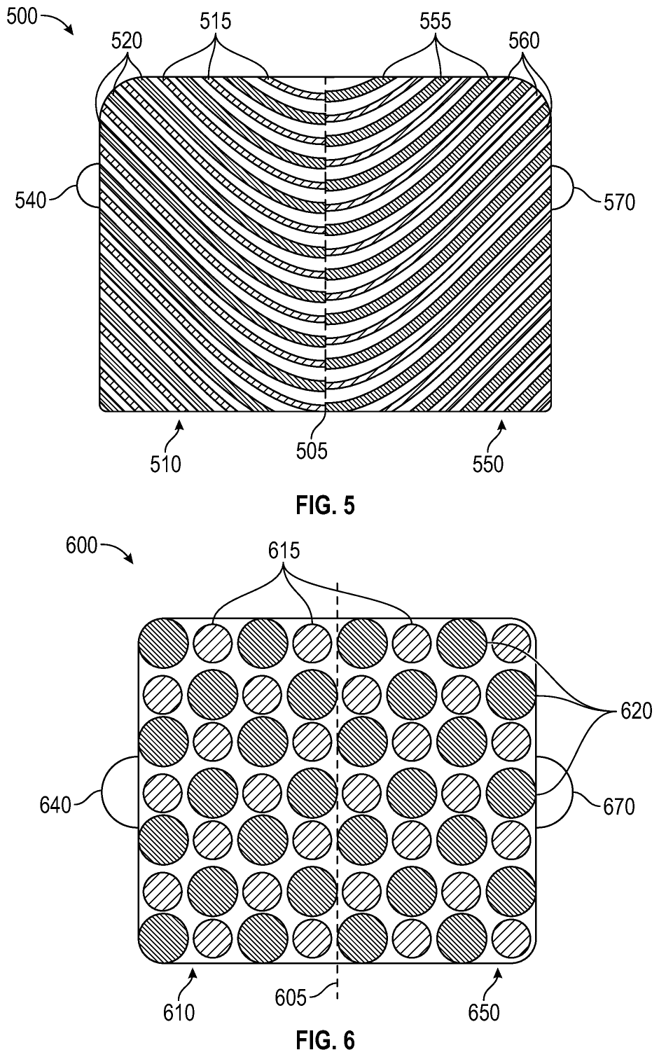

FIG. 5 is an illustration of one embodiment of a face mask with a patterned engagement structure as disclosed herein. As shown in FIG. 5, the face mask 500 may comprise a left half 510 and a right half 550 divided by a center axis 505 and tabs 540, 570. The PSA portions 515, 560 and release-coating portions 520, 555 of the face mask 500 may function substantially similarly to the PSA portions 315, 360, 365 and release-coating portions 320, 325, 355 of the face mask 300 shown in FIG. 3, except that the PSA portions 515, 560 and release-coating portions 520, 555 of the face mask 500 may cover a much larger portion of the interior of the face mask 500 than some other embodiments.

FIG. 6 is an illustration of one embodiment of a transdermal patch with a patterned engagement structure as disclosed herein. As shown in FIG. 6, the transdermal patch 600 may comprise a left half 610 and a right half 650 divided by a center axis 605, and tabs 640, 670. The PSA portions 615 and release-coating portions 620 of the transdermal patch 600 may function substantially similarly to the PSA portions 515, 560, 565 and release-coating portions 520, 525, 555 of the face mask 500 shown in FIG. 5, except that the PSA portions 615 and release-coating portions 620 of the transdermal patch 600 may be circle in shape, wherein the PSA portions 615 may be slightly smaller in size than the release-coating portions 620.

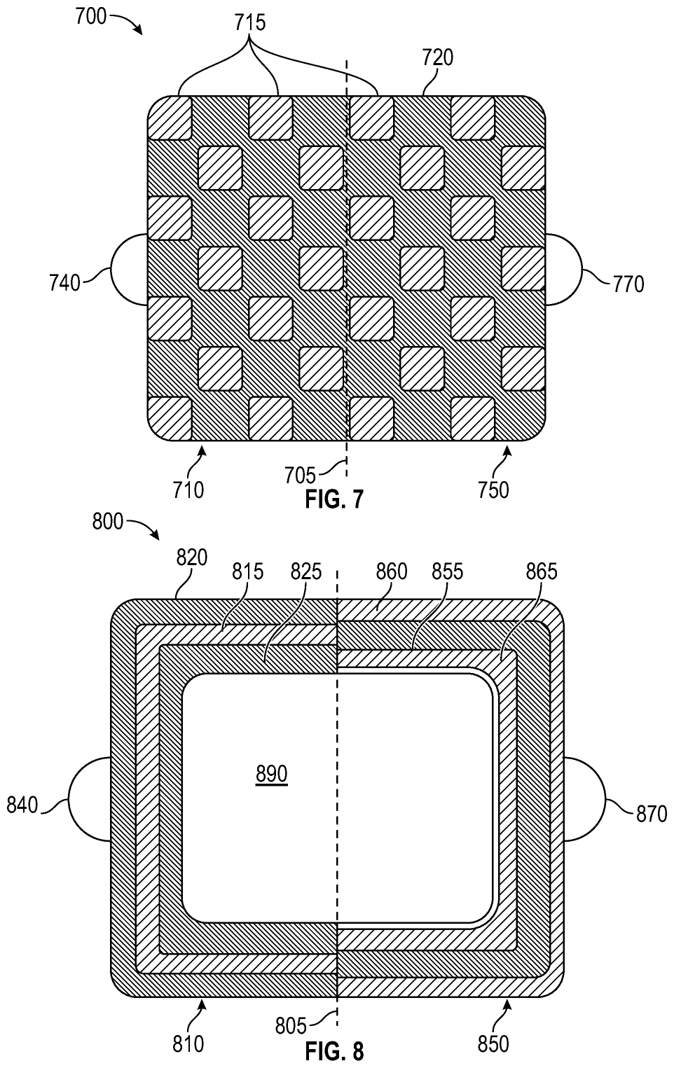

FIG. 7 is an illustration of one embodiment of a transdermal patch with a patterned engagement structure as disclosed herein. As shown in FIG. 7, the transdermal patch 700 may comprise a left half 710 and a right half 750 divided by a center axis 705, and tabs 740, 770. The PSA portions 715 and release-coating portion 720 of the transdermal patch 700 may function substantially similarly to the PSA portions 615, 670, 675 and release-coating portions 620, 625, 655 of the transdermal patch 600 shown in FIG. 6, except that the PSA portions 715 comprise rounded squares, while the release-coating portion 720 of the transdermal patch 700 may be substantially the entire interior of the surface that is not the PSA portions 715.

FIG. 8 is an illustration of one embodiment of a transdermal patch with a patterned engagement structure as disclosed herein. As shown in FIG. 8, the transdermal patch 800 may comprise a left half 810 and a right half 850 divided by a center axis 805, tabs 840, 870, and a medicated surface 890. The PSA portions 815, 860, 865 and release-coating portions 820, 825, 855 of the transdermal patch 800 may function substantially similarly to the PSA portions 315, 360, 365 and release-coating portions 320, 325, 355 of the face mask 300 shown in FIG. 3. Additionally, the medicated surface 890 may comprise any medication or formulation that may be desired to be absorbed through the skin. One benefit of the transdermal patch 800 disclosed herein is that the transdermal patch may be reused, or have used time shortened, stored, and then reused depending on the qualities of the medicated surface 890. Additionally, the medicated surface 890 may be sealed against itself, such that none of the medication or formulation is lost during unpackaging or application to a wearer's skin.

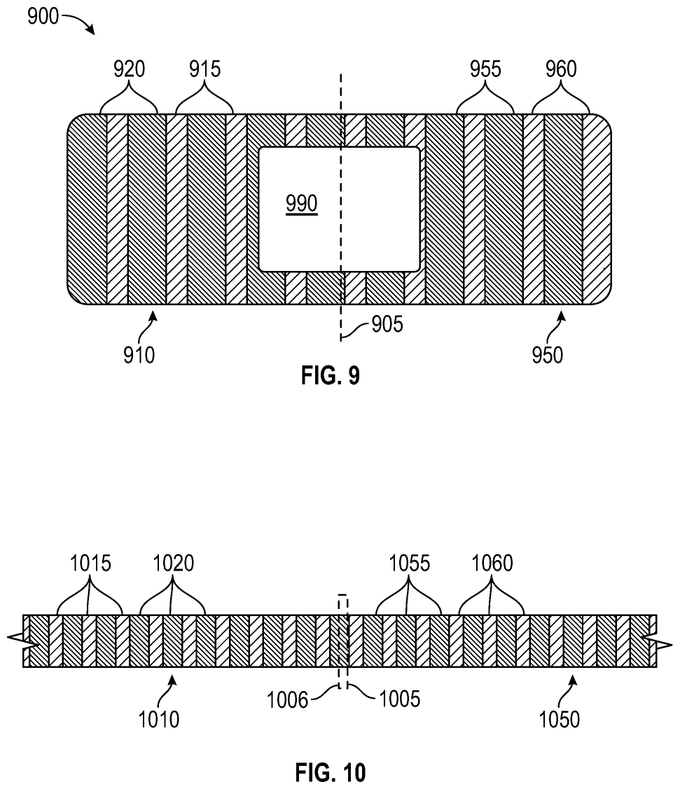

FIG. 9 is an illustration of one embodiment of an adhesive bandage with a patterned engagement structure as disclosed herein. As shown in FIG. 9, the adhesive bandage 900 may comprise a left half 910 and a right half 950 divided by a center axis 905, and an injury surface 990. The PSA portions 915, 960 and release-coating portions 920, 955 of the adhesive bandage 900 may function substantially similarly to the PSA portions 515, 560 and release-coating portions 520, 555 of the face mask 500 shown in FIG. 5, and may also be a series of parallel strips. In some embodiments, if folded not along the center axis 905, the adhesive bandage 900 may be permanently sealed for ease of disposal of injury related waste. Additionally, the injury surface 990 may be configured to cover a minor injury and facilitate healing by protecting the minor injury from outside contaminants. In a preferred embodiment, the injury surface 990 may be configured to not stick or attach to the user's injury or other skin.

FIG. 10 is an illustration of one embodiment of a reusable tape with a patterned engagement structure as disclosed herein. As shown in FIG. 10, the reusable tape 1000 may comprise a left half 1010 and a right half 1050 divided by a center axis 1005. The PSA portions 1015, 1060 and release-coating portions 1020, 1055 of the reusable tape 1000 may function substantially similarly to the PSA portions 515, 560 and release-coating portions 520, 555 of the face mask 500 shown in FIG. 5. Additionally, the reusable tape may also comprise a permanent sealing axis 1006, such that if the reusable tape is folded along the permanent sealing axis 1006, the PSA portions 515, 560 are configured to engage one another and create a stronger, potentially permanent bond. In a preferred embodiment, the reusable tape 1000, when folded along the center axis 1005, may be folded and unfolded repeatedly, thereby allowing the tape to be reused multiple times.

In various embodiments, the material used in the PSA portions of each of the figures disclosed herein may be washed or cleaned to allow the masks to be reused and function as multi-use masks.

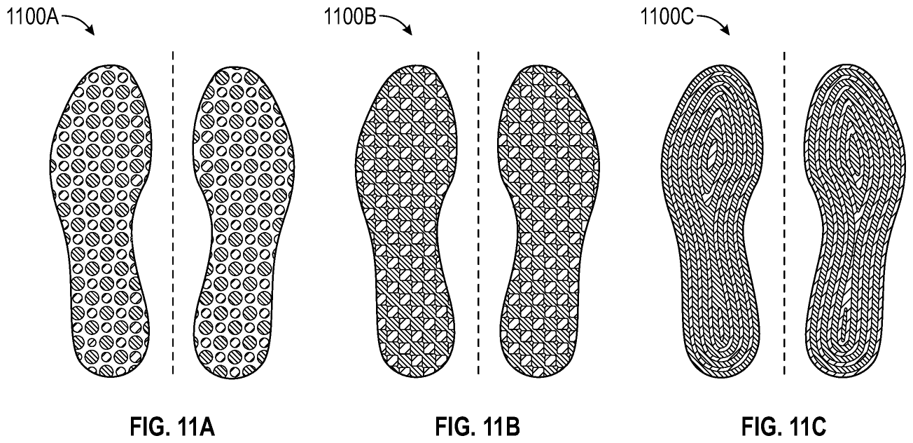

FIGS. 11A-11C are illustrations of embodiments of adhesive shoes with a patterned engagement structure as disclosed herein. The adhesive shoes 1100A, 1100B, 1100C each have PSA portions and release-coating portions configured to engage one another such that the two surfaces that constitute a pair of adhesive shoes 1100A, 1100B, 1100C may be stored together in a compact fashion. When the two surfaces that constitute a pair of adhesive shoes 1100A, 1100B, 1100C are separated, they may be affixed to a wearer's feet, and act as shoes.

The versatility of the patterned engagement structure enables application to a variety of uses. For example, this disclosure may be applicable to both bilateral/inverted (near) symmetry and a central fold (e.g., adhesive face mask), as well as inverted (near) symmetry of separate paired and non-paired pieces (e.g., adhesive shoes, double-sided tape). However, in other embodiments, products may be asymmetrical—the inverted (near) symmetry of the PSA/release-coating patterns may only occupy a portion of the product (e.g., envelope), or the inverted (near) symmetry may only become evident when a discrete section of the product is taken into consideration (e.g., a short piece of continuous tape).

Thus, the disclosure may be applicable to various categories of embodiments. One category goes to release-liner-free, single-use disposable products. Embodiments of this category provide an alternative to single-use disposable products that use traditional release liners. Omitting the need for a release liner provides a more-convenient and less-wasteful product. Another category goes to release liner-free, multi-use, disposable embodiments, which improves upon single-use disposable products by making them easier to use and reuse repeatedly, improving their convenience, extending their functional longevity, and further reducing wastes from single-use disposables. Another category goes to release liner-free, single-use, durable embodiments, which comprises durable products (e.g., roofing membranes) that are applied once and remain applied for an extended period of time. Another category goes to release liner-free, reusable, durable embodiments, which comprises products designed for long-term repeated use and make use of washable adhesives with renewable tack (e.g., nano-PU), durable release coatings/materials, and durable substrates (e.g., cloth rather than paper).

In embodiments of the above categories, the patterned engagement structure may be applicable to other products. For example, the patterned engagement structure may be applied to: diagnostic testing components such as sensors, electrodes, and grounding pads; fixation devices such as catheters or tubing; transdermal medication patches; eye patches; bandages; diapers; incontinence pads; sanitary napkins; under-eye masks; facial patches; nail guards; stick-on nails; nasal and mouth strips, such as for snoring or sports; kinesiology tape strips; stick-on body protection such as elbow pads, knee pads, and hand protection; blister/wound protection; multi-use tape roll or tape strips; resealable envelopes, containers, pouches, or boxes; thermoformed, blister, and clamshell packaging; labeling such as post-it notes, labels, luggage tags, windshield stickers; container lids; bags or pouches; wearable sensors; ticketing; tracking wearables; wireless headphones; gloves; bras; pasties; jewelry; body safety reflectors; vehicle tracking; waterproofing existing products or roofing products; wallpaper; decals; erector sets; temporary tattoos; sun-visors; movie glasses; patches for inflatables; and wall hooks.

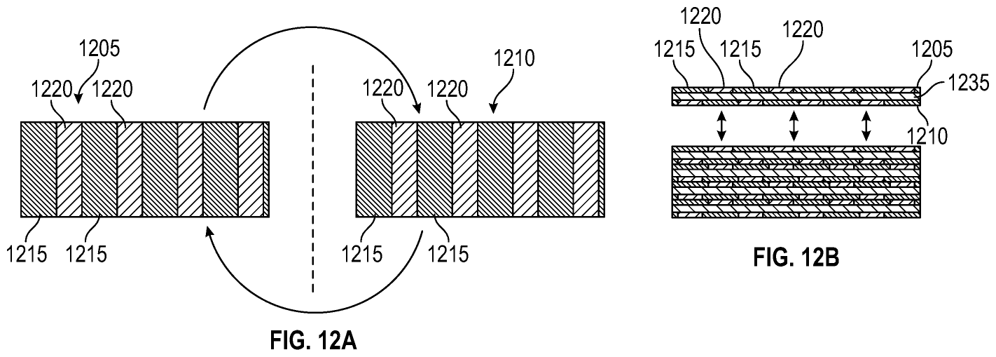

FIGS. 12A and 12B are illustrations of a double-sided tape with a patterned engagement structure as disclosed herein. As shown in FIG. 12A, a double-sided tape may comprise a top portion 1205 and a bottom portion 1210. Both the top portion 1205 and the bottom portion 1210 comprise alternating patterns of release-coating portions 1215 and PSA portions 1220. As shown in FIG. 12B, the double-sided tape may further comprise an inner portion 1235 in between the top portion 1205 and the inner portion 1210, wherein the top portion 1205 is connected to one side of the inner portion 1235 and the bottom portion 1210 is connected to the other side of the inner portion 1235. The inner portion 1235 may be made of any material, such as carrier paper or film. As is also shown in FIG. 12B, the multiple double-sided tapes may be stacked on top of each other.

What is claimed is:

1. A face mask, comprising:
an inner left half and an inner right half;
one or more adhesive portions; and
one or more release-coating portions, wherein the one or more release-coating portions comprise material that has been cured;
wherein the left inner half and the right inner half are divided by a center axis.

2. The face mask of claim 1, wherein the adhesive portions and the release-coating portions are located along a section of the inner left half; and
wherein the adhesive portions and the release-coating portions are located along a section of the inner right half;
wherein the location of the adhesive portions of the inner left half substantially mirror the location of the release-coating portions of the inner right half along the center axis; and
wherein the location of the release-coating portions of the inner left half substantially mirror the location of the adhesive portions of the inner right half along the center axis.

3. The face mask of claim 2, comprising a symmetrical folded configuration and an unfolded configuration;
wherein in the unfolded configuration, the adhesive portions of the inner left half and the adhesive portions of the inner right half are configured to releasably engage a surface.

4. The face mask of claim 3, wherein when the face mask is in the symmetrical folded configuration, the adhesive portions of the inner left half engage the release-coating portions of the inner right half.

5. The face mask of claim 4, further comprising an asymmetrical folded configuration formed by folding the inner left half and the inner right half along an axis such that at least two or more of the adhesive portions engage one another.

6. The face mask of claim 2, further comprising a liner configured to cover at least the adhesive portions.

7. The face mask of claim 2, further comprising a left tab and a right tab, wherein the left tab extends from the left half, and wherein the right tab extends from the right half.

8. The face mask of claim 2, wherein the adhesive portions are washable and wherein the face mask is reusable.

9. A patterned engagement structure, comprising:
two or more surfaces;
wherein the two or more surfaces comprise a first surface and a second surface;
wherein the first surface comprises one or more first adhesive portions and one or more first release-coating portions and the one or more first adhesive portions of the first surface are located between or to at least one side of the one or more first release-coating portions of the first surface; and
wherein the second surface comprises one or more second adhesive portions and one or more second release-coating portions and the one or more second adhesive portions of the second surface are located between or to at least one side of the one or more second release-coating portions of the second surface.

10. The patterned engagement structure of claim 9, wherein the first surface and the second surface are configured to releasably engage one another such that the first adhesive portions are configured to substantially engage the second release-coating portions and the second adhesive portions are configured to substantially engage the first release-coating portions.

11. The patterned engagement structure of claim 10, wherein portions of the first surface comprising the one or more first release-coating portions substantially include the one or more first release-coating portions but not the one or more first adhesive portions, and other portions of the first surface comprising the one or more first adhesive portions substantially include the one or more first adhesive portions but not the one or more first release-coating portions, and wherein portions of the second surface comprising the one or more second release-coating portions substantially include the one or more second release-coating portions but not the one or more second adhesive portions, and other portions of the second surface comprising the one or more second adhesive portions substantially include the one or more second adhesive portions but not the one or more second release-coating portions.

12. The patterned engagement structure of claim 11, wherein the first surface and the second surface are each a portion of a single continuous material, and wherein the single continuous material is configured to fold along a central axis such that the first adhesive portions are configured to substantially engage the second release-coating portions and the second adhesive portions are configured to substantially engage the first release-coating portions.

13. The patterned engagement structure of claim 12, wherein the adhesive portions are pressure sensitive.

14. The patterned engagement structure of claim 13, wherein the adhesive portions is made up of at least one of a rubber-based adhesive, a silicone-based adhesive, an acrylate-based adhesive, a synthetic adhesive, a nano-PU-based, a hydrocolloids adhesive, a hydrogel adhesive, a PLA-based pressure-sensitive adhesive, or a PHA-based pressure-sensitive adhesive.

15. The patterned engagement structure of claim 13, wherein the release-coating portions is made up of at least one of a silicone material, a fluorosilicone material, a synthetic material, a non-biodegradable material, a plastics-free material, or a biodegradable material.

16. The patterned engagement structure of claim 13, further comprising a third surface with a top and a bottom, wherein the third surface is positioned between the first surface and the second surface such that the first surface is attached to the top of the third surface and the second surface is attached to the bottom of the third surface.

17. The patterned engagement structure of claim 13, wherein the two or more surfaces are flexible.

18. A face mask, comprising:
an inner left half and an inner right half;
one or more adhesive portions; and
one or more release-coating portions comprising material that has been cured;
wherein the inner left half and the inner right half are divided by a center axis;
wherein the adhesive portions are located in a section of the inner left half and in a section of the inner right half;
wherein the face mask comprises a symmetrical folded configuration and an unfolded configuration;
wherein the inner left half and the inner right half are configured to releasably engage one another in the symmetrical folded configuration;
wherein the inner left half and the inner right half are configured to releasably engage a surface in the unfolded configuration; and
wherein the location of the adhesive portions and the location of the release-coating portions substantially mirror each other along the center axis such that when the face mask is in the symmetrical folded configuration, the adhesive portions engage the release-coating portions.

19. The face mask of claim 18, wherein a size of the adhesive portions is either smaller or bigger than a size of the release-coating portions to which the adhesive portions are configured to engage when in the symmetrical folded configuration.

20. The face mask of claim 18, wherein the adhesive portions are washable and wherein the face mask is reusable.
